(19) United States
(12) Patent Application Publication
(10) Pub. No.: US 2005/0033139 A1
(43) Pub. Date: Feb. 10, 2005

(54) Adaptive segmentation of anatomic regions in medical images with fuzzy clustering

(75) Inventors: Ruiping Li, Rockville, MD (US); Xin-Wei Xu, Gaithersburg, MD (US); Jyh-Shyan Lin, North Potomac, MD (US); Fleming Y.-M Lure, Potomac, MD (US); H.-Y Michael Yeh, Potomac, MD (US)

Correspondence Address: VENABLE, BAETJER, HOWARD AND CIVILETTI, LLP, P.O. BOX 34385, WASHINGTON, DC, 20043-9998, US

(73) Assignee: Deus Technologies, LLC, Rockville, MD (US)

(21) Appl. No.: 10/606,120

(22) Filed: Jun. 26, 2003

Related U.S. Application Data
(60) Provisional application No. 60/394,238, filed on Jul. 9, 2002.

(51) Int. Cl. A61B 5/05

(52) U.S. Cl. 600/407

(57) ABSTRACT

A method for identifying the orientation of an interesting object in a digital medical image comprises steps of creating a rectangular interesting image mask that covers the interesting object, based on the original digital medical image; generating a rough image based on the interesting image mask, the rough image coarsely describing the interesting object; and identifying the orientation of the interesting object based on the rough image. A method for segmenting interesting objects in digital medical images may also comprise steps of creating a rectangular interesting image mask that covers said interesting object, based on an original digital medical image; generating a rough image based on the interesting image mask, the rough image coarsely describing the interesting object; and performing a post-process on the rough image.

### CROSS-REFERENCE TO RELATED APPLICATION

This application claims the benefit of U.S. Provisional Application No. 60/394,238, filed Jul. 9, 2002, and incorporated herein by reference.

### BACKGROUND OF THE INVENTION

1. Field of the Invention

The present invention relates to anatomic region-based medical image processing and relates to automated detection of human diseases. It more specifically relates to computer-aided detection (CAD) methods for automated detection of lung nodules in chest images, such as segmentation of anatomic regions in chest radiographic images and identification of orientation of postero-anterior (PA) chest images using fuzzy clustering techniques.

2. Background Art

Lung cancer is the leading type of cancer in both men and women worldwide. Early detection and treatment of localized lung cancer at a potentially curable stage can significantly increase the patient survival rate.

Among the common detection techniques for lung cancer, such as chest X-ray, analysis of the types of cells in sputum specimens, and fiber optic examination of bronchial passages, chest radiography remains the most effective and widely used method. Although skilled pulmonary radiologists can achieve a high degree of accuracy in diagnosis, problems remain in the detection of the lung nodules in chest radiography due to errors that cannot be corrected by current methods of training, even with a high level of clinical skill and experience.

Studies have shown that approximately 68% of retrospectively detected lung cancers were detected by one reader and approximately 82% were detected with an additional reader as a “second-reader.” A long-term lung cancer screening program conducted at the Mayo Clinic found that 90% of peripheral lung cancers were visible in small sizes in retrospect, in earlier radiographs.

An analysis of human error in the diagnosis of lung cancer revealed that about 30% of the missed detections were due to search errors, about 25% were due to recognition errors, and about 45% were due to decision-making errors. (Reference is made to Kundel, H. L, et al., “Visual Scanning, Pattern Recognition and Decision-Making in Pulmonary Nodule Detection”, Investigative Radiology, May-June 1978, pp 175-181, and Kundel, H. L., et al., “Visual Dwell Indicates Locations of False-Positive and False-Negative Decisions”, Investigative Radiology, June 1989, Vol. 24, pp 472-478, which are incorporated herein by reference.) The analysis suggests that the miss rates for the detection of small lung nodules could be reduced by about 55% with a computerized method. According to the article by Stitik, F. P., “Radiographic Screening in the Early Detection of Lung Cancer”, Radiologic Clinics of North America, Vol. XVI, No. 3, December 1978, pp 347-366, which is incorporated herein by reference, many of the missed lesions would be classified as T1M0 lesions, the stage of non-small cell lung cancer that Mountain, C. F., “Value of the New TNM Staging System for Lung Cancer”, 5th World Conference in Lung Cancer Chest, 1989 Vol. 96/1, pp 47-49, which is incorporated herein by reference, indicates has the best prognosis (42%, 5 year survival). It is this stage of lung cancer, with lesions smaller than 1.5 cm in diameter, and located outside the hilum region, that needs to be detected by a radiologist in order to improve survival rates.

Computerized techniques, such as computer-aided detection (CAD), have been introduced to assist in the detection of lung nodules during the early stage of non-small cell lung cancer. The CAD technique requires the computer system to function as a second reader to double-check the films that a primary physician has examined. An exemplary automated system for the detection of lung nodules may include five functional units. They are:
lung segmentation,
initial selection of suspect nodules,
feature generation of nodules,
reduction of false positives (e.g., classification), and
decision unit.
   (See, e.g., U.S. patent application Ser. No. 09/625,418 to Li et al., entitled “Fuzzy logic based classification (FLBC) method for automated identification of nodules in radiological images,” filed on Jul. 25, 2000, currently pending; U.S. patent application Ser. No. 09/018,789 to Lure et al., entitled “Method and system for re-screening nodules in radiological images using multi-resolution processing, neural network, and image processing,” filed on Oct. 12, 1999, now abandoned; and U.S. patent application Ser. No. 09/503,840 to Lin et al., entitled, “Divide-and conquer method and system for the detection of lung nodule in radiological images,” filed on Feb. 20, 2000, currently pending; all of which are commonly assigned and hereby incorporated by reference in their entireties.)

Obviously, identification of the lung field location is a top priority in lung nodule detection. Although a number of computer algorithms have been developed to automatically identify the lung regions in a digitized postero-anterior chest radiograph (DCR), they can be generally described as either edge-based or area-based in terms of methodology. Typically, the edge-based lung region segmentation approach has been described in the following references: J. Duryea and M. Boone, “A fully automated algorithm for the segmentation of lung fields on digital chest radiographic images,” Med. Phys. 22, pp 183-191, 1995; X.-W. Xu and K. Doi, “Image features analysis for computer-aided diagnosis: Accuracy determination of ribcage boundary in chest radiographs,” Med. Phys. 22, pp 617-626, 1995; and F. M. Carrascal, J. M. Carreira, M. Souto, P. G. Tahoces, L. Gornez, and J. J. Vidal, “Automatic calculation of total lung capacity from automatically traced lung boundaries in postero-anterior and lateral digital chest radiographs,” Med. Phys. 25, pp 11 18-1131, 1998.

This edge-based approach detects the edge lines in profiles, signatures, or kernels of its original two-dimensional image. Duryea et al. presented an automated algorithm to identify both lungs on digital chest radiographs. Starting points for the edge tracing process, which provided four edges, i.e., upper-medial, upper-lateral, lower-medial, and lower-lateral edges, were extracted based on horizontal profiles. The algorithm was evaluated with 802 images. The average accuracies were 95.7% for the right lung and 96.0% for the left lung.

Xu et al. developed a computerized method for automated determination of ribcage boundaries in digital chest radiographs. The average position of the top of the lung was determined based on the vertical profile and its first derivative in the upper central area of the chest image. Top lung edges and rib cage edges were determined within search ROIs (regions of interest), which were selected over top lung cages and rib cage. The complete rib cage boundary was obtained by smoothly connecting three curves. Xu et al. used a subjective evaluation to examine the accuracy of the results for 1000 images. The overall accuracy of the method was 96% based on the evaluations of five observers.

Carrascal et al. developed an automated computer-based method for the calculation of total lung capacity (TLC), by determining the pulmonary contours from digital PA and lateral radiographs of the thorax. This method consists of four main steps: 1) determining a group of reference lines in each radiograph; 2) defining a family of rectangular ROIs, which include the pulmonary borders, in each of which the pulmonary border is identified using edge enhancement and thresholding techniques; 3) removing outlying points from the preliminary boundary set; and 4) correcting and completing the pulmonary border by means of interpolation, extrapolation, and arc fitting. The method was applied to 65 PA chest images. Three radiologists carried out a subjective evaluation of the automatic tracing of the pulmonary borders with use of a five-point rating scale. The results were 44.1% with a score of 5, 23.6% with a score of 4, 7.2% with a score of 3, 19.0% with a score of 2, and 6.1% with a score of 1.

On the other hand, the approach of area-based lung region segmentation usually uses image features, such as density (pixel gray level), histogram, entropy, gradients, and co-occurrence matrix to perform classification. In the existing methods, the techniques used include neural network techniques and discriminant analysis, examples of which follow, and which are incorporated herein by reference.

M. F. McNitt-Gray, H. K. Huang, and J. W. Sayre, “Feature selection in the pattern classification problem of digital chest radiograph segmentation”, IEEE Trans. Med. Images 14, pp 537-547, 1995, employed a linear discriminator and a feed-forward neural network to classify pixels in a digital chest image into five areas using a set of image features selected. The five areas represent five different anatomic regions: 1) includes heart, subdiaphragm, and upper mediastinum; 2) includes right and left lungs; 3) includes two side axillas; 4) includes base of head/neck; and 5) is the background, which includes the area outside the patient projection but within the radiation field. McNitt-Gray et al. introduced a list of candidate features that include gray-level-based features, measures of local differences, and measures of local texture. A feature selection step was used to choose a subset of features from the list of candidate features. The number of nodes in the input layer was determined by the number of features in the subset. The neural network classifier was trained by using back-propagation learning.

Hasegawa, S.-C. Lo, J.-S. Lin, M. T. Freedman, and S. K. Mun, “A shift invariant neural network for the lung field segmentation in chest radiography”, J. of VLSI Signal Processing 18, pp 241-250, 1998, developed a computerized method using a shift-invariant neural network for the segmentation of lung fields in chest radiography. Only pixel gray levels served as inputs of the neural network. The lung fields were extracted by employing a shift-invariant neural network that used an error back-propagation training method. In order to train the neural network, Hasegawa et al. generalized the corresponding reference image in advance for each of the training cases. In their study, a set of computer algorithms was also developed for smoothing the initially-detected edges of lung fields. The results indicated that 86% of the segmented lung fields globally matched the original chest radiographs for 21 testing images.

Tsujii, M. T. Freedman, and S. K. Mun, “Automated segmentation of anatomic regions in chest radiographs using an adaptive-sized hybrid neural network”, Med. Phys. 25, pp 998-1007,1998, developed an automated computerized method for lung segmentation. In contrast with the method of Hasegawa et al., Tsujii et al. chose four image features as inputs of the neural network; these are relative addresses (Rx, Ry), normalized density, and histogram equalized entropy. The network was trained using 14 images. The trained neural network classified lung regions with 92% accuracy when compared against the 71 test images following the same rules used for the training images.

N. F. Vittitoe, R. Vargas-Voracek, and Carey E. Floyd, Jr., “Markov random field modeling in postero-anterior chest radiograph segmentation”, Med. Phys. 26, pp 1670-1677, 1999, presented an algorithm to identify multiple anatomical regions in a digitized PA chest radiograph utilizing Markov random field (MRF) modeling. The MRF model was developed using 115 chest radiographs. An additional 115 chest radiographs served as a test set. On average for the test set, the MRF technique correctly classified 93.3% of the lung pixels, 89.8% of the subdiaphragm pixels, 78.3% of the heart pixels, 86.1% of the mediastinum pixels, 90.1% of the body pixels, and 88.4% of the background pixels.

Unfortunately, direct comparison of the performance of these various techniques can not be made because of the differences in the data sets. A common point of uncertainty with these experiments is universality of the specific data set used. If a method is tested using 1000 “similar” images, the meaning of the calculated accuracy is limited. Generally, different digitizers, different patients and different film-makers should affect the accuracy of a method of analysis. Additionally, the existing methods do not deal with identification of orientation of PA chest images. None of these methods simultaneously considers how to provide useful information for classification of lung nodules while segmenting lung regions.

### SUMMARY OF THE INVENTION

Accordingly, one object of this invention is to provide a novel segmentation method, based on fuzzy clustering, and a set of specified post-processing techniques, which include noise reduction, determination of top and bottom points of lung, border detection, boundary smoothing, and modification of regions, for automated identification of anatomic regions in chest radiographs.

Another object of this invention is to provide a novel identification method for the detection of orientation of PA chest radiographs that may be oriented in either portrait or landscape view.

The invention further enables the detection of indicators of lung diseases, such as lung nodules. The invention also can be used for other areas, including but not limited to (1) breast tumor detection, (2) brain MRI segmentation, (3) interstitial lung disease classification, (4) CT image segmentation, (5) microcalcification identification, and (6) anatomic-region-based image processing.

Additionally, the invention may be embodied as a computer programmed to carry out the inventive method, as a storage medium storing a program for implementing the inventive method, and as a system for implementing the method.

These and other objects are achieved according to an embodiment of the present invention by providing a new method for segmenting anatomic regions in a digitized PA chest image and identifying orientation of a digitized PA chest image, including (a) subsampling image data obtained in order to speed up the computational process; (b) performing a fuzzy clustering algorithm for the subsampled image data and thus generating a rough image; (c) subjecting the rough image to a filter that is designed to assimilate isolated points in each region; (d) identifying the orientation of original chest image based on the rough image after step (c); (e) determining lung's top points and bottom points in the rough image; (f) detecting the border points of each region; (g) smoothing the boundaries of each region; and (h) adjusting the boundaries of each region based on human experience.

According to an embodiment of the invention, step (a) includes setting a reduction factor of image size to two to obtain a 263×319 image from an original 525×637 image.

According to an embodiment of the invention, step (b) includes performing a Gaussian clustering algorithm for the subsampled image data to generate a rough image in which pixels are classified into several classes based on pixel gray level.

Preferably, the employed Gaussian clustering method of step (b) includes performing self-organizing classification for pixels under a predetermined number of classes, where training or prior knowledge is unnecessary. Moreover, the process maybe fully automatic, and parameters need not be problem-specific.

According to a preferred embodiment of the invention, step (c) includes using a 3×3 table filter to assimilate isolated points in each class.

According to an embodiment of the invention, step (d) includes identifying the orientation of the original chest image based on the rough image generated by step (c). Preferably, the orientation identification method of step (d) further includes detecting a midline landmark of the chest image and determining a boundary of the central zone which includes most of the superior mediastinum (Sms), most of the heart (Hrt) area, and part of the subdiaphragm (Sub).

According to an embodiment of the invention, step (e) includes detecting the outer point of top lung (OTL), inner point of top lung (ITP), outer point of bottom lung (OBL), and inner point of bottom lung (IBL) for both right lung and left lung, based on the obtained rough image.

According to an embodiment of the invention, step (f) includes detecting the border points of the lung region, using information of top lung points and bottom lung points that were detected in step (e), based on the rough image, from the top of the lung to the bottom of the lung.

According to an embodiment of the invention, step (g) includes using heuristic rules based on spatial information to smooth the boundaries of the lung zones. In this step, two processes are preferably used. These are top-down trimming and bottom-up trimming. The former serves (1) to cut the connection between the top lung and the shoulder, and (2) to cut the connection between the bottom lung and the background, if applicable. The latter is designed to refill any part of the lung region that is misclassified. Preferably, the boundary obtained by using this bidirectional trimming method is not only smooth but also natural.

Preferably, step (h) of an embodiment of the inventive method includes using a set of empirical parameters that are determined by testing an entire training image data set to adjust the area of each of the regions, through extension and/or shrinking of the boundaries.

Definitions

In describing the invention, the following definitions are applicable throughout (including above).

A “computer” refers to any apparatus that is capable of accepting a structured input, processing the structured input according to prescribed rules, and producing results of the processing as output. Examples of a computer include: a computer; a general purpose computer; a supercomputer; a mainframe; a super mini-computer; a mini-computer; a workstation; a microcomputer; a server; an interactive television; a hybrid combination of a computer and an interactive television; and application-specific hardware to emulate a computer and/or software. A computer can have a single processor or multiple processors, which can operate in parallel and/or not in parallel. A computer also refers to two or more computers connected together via a network for transmitting or receiving information between the computers. An example of such a computer includes a distributed computer system for processing information via computers linked by a network.

A “computer-readable medium” refers to any storage device used for storing data accessible by a computer. Examples of a computer-readable medium include: a magnetic hard disk; a floppy disk; an optical disk, like a CD-ROM or a DVD; a magnetic tape; a memory chip; and a carrier wave used to carry computer-readable electronic data, such as those used in transmitting and receiving e-mail or in accessing a network.

“Software” refers to prescribed rules to operate a computer. Examples of software include: code segments; instructions; computer programs; and programmed logic.

A “computer system” refers to a system having a computer, where the computer comprises a computer-readable medium embodying software to operate the computer.

A “network” refers to a number of computers and associated devices that are connected by communication facilities. A network involves permanent connections such as cables or temporary connections such as those made through telephone or other communication links. Examples of a network include: an internet, such as the Internet; an intranet; a local area network (LAN); a wide area network (WAN); and a combination of networks, such as an internet and an intranet.

### BRIEF DESCRIPTION OF THE DRAWINGS

The features of the present invention and the manner of attaining them will become apparent, and the invention itself will be understood, by reference to the following description and the accompanying drawings, wherein:

FIG. 1 is a block diagram of an embodiment of the present invention for segmenting lung regions in digitized chest radiographic images;

FIG. 2 is a 525×637 chest portrait image digitized from X-ray film;

FIG. 3 is a 525×637 chest landscape image digitized from X-ray film;

FIG. 4 is a flow chart illustrating steps in an embodiment of the preprocessing unit of FIG. 1;

FIG. 5 is a flow chart illustrating steps in an embodiment of the fuzzy clustering unit of FIG. 1;

FIG. 6 is a rough image generated by the fuzzy clustering unit of FIG. 1, based on the chest image of FIG. 2;

FIG. 7 is a PA chest portrait image identified by the orientation identification unit of FIG. 1 through finding the spinal area in the vertical direction;

FIG. 8 is a PA chest landscape image identified by the orientation identification unit of FIG. 1 through finding the spinal area in the horizontal direction;

FIG. 9 is a block diagram of an embodiment of the post-processing unit of FIG. 1;

FIG. 10 is the result of the image of FIG. 7 being processed by the isolated-point assimilation block of FIG. 9;

FIG. 11 shows the outer point of top lung (OTL) classified in Type 1, inner point of top lung (ITL), and outer point of bottom lung (OBL) classified in Case 1, where the inner point of bottom lung (IBL) here is in the same location as OBL;

FIG. 12 shows the inner point of top lung (ITL) classified in Type 2, and outer point of bottom lung (OBL) classified in Case 1, where OTL is the same as ITL and IBL is the same as OBL;

FIG. 13 shows the outer point of bottom lung (OBL) classified in Case 2;

FIG. 14 is a result after processing the right lung of FIG. 11 by the top-down trimming part of FIG. 9;

FIG. 15 is a result after processing the right lung of FIG. 14 by the bottom-up trimming block of FIG. 9;

FIG. 16 is an initial mask image that was obtained by passing the image of FIG. 11 through the top-down trimming block and the bottom-up trimming block;

FIG. 17 is a final zone mask image that was obtained by passing the initial mask image of FIG. 16 through the extension/shrink block of FIG. 9;

FIG. 18 is an image that was obtained by letting the portrait image of FIG. 2 overlay boundaries of the zone mask image of FIG. 17;

FIG. 19 is an image that was obtained by letting the landscape image of FIG. 3 overlay boundaries of the corresponding zone mask image;

FIG. 20 is a mask image that was obtained by applying the same concept to a 2-D CT image; and

FIG. 21 is the corresponding original 2-D CT overlaying boundaries of the lung mask image of FIG. 20.

### DETAILED DESCRIPTION OF THE PREFERRED EMBODIMENTS

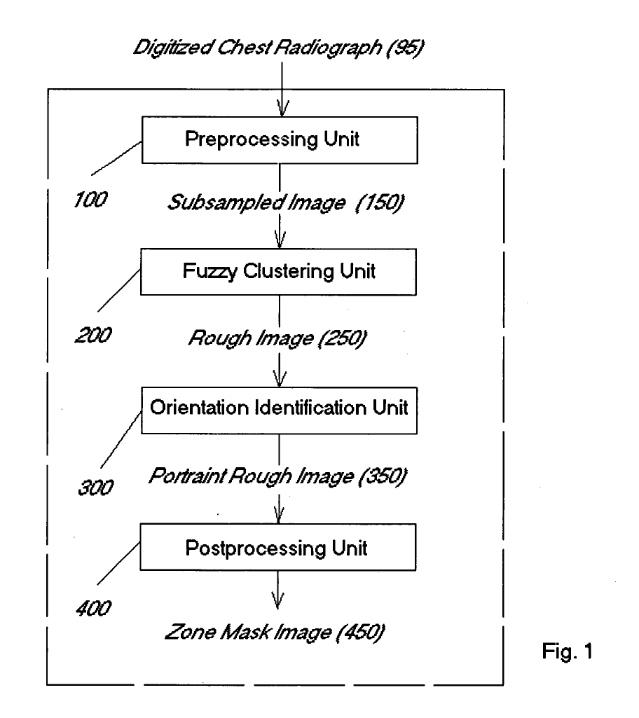

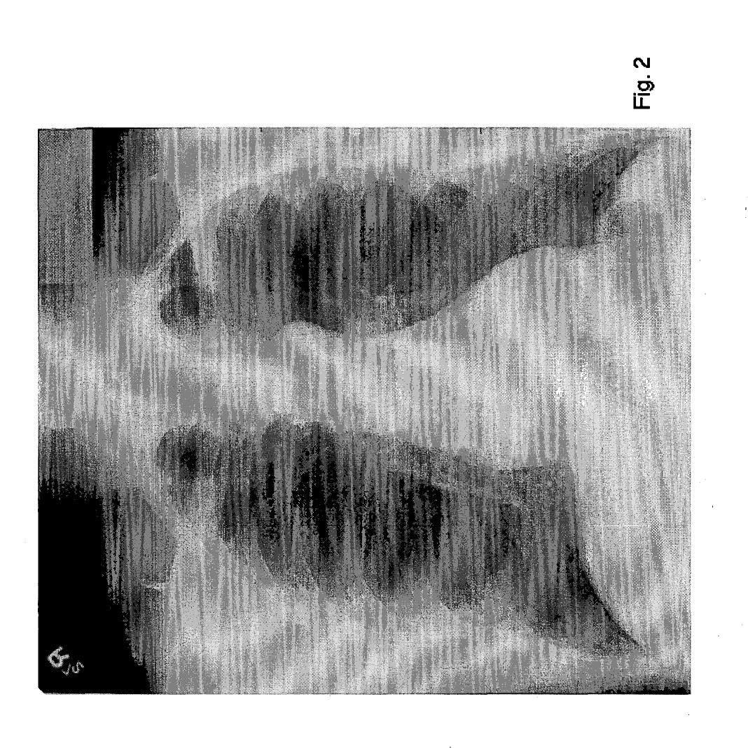

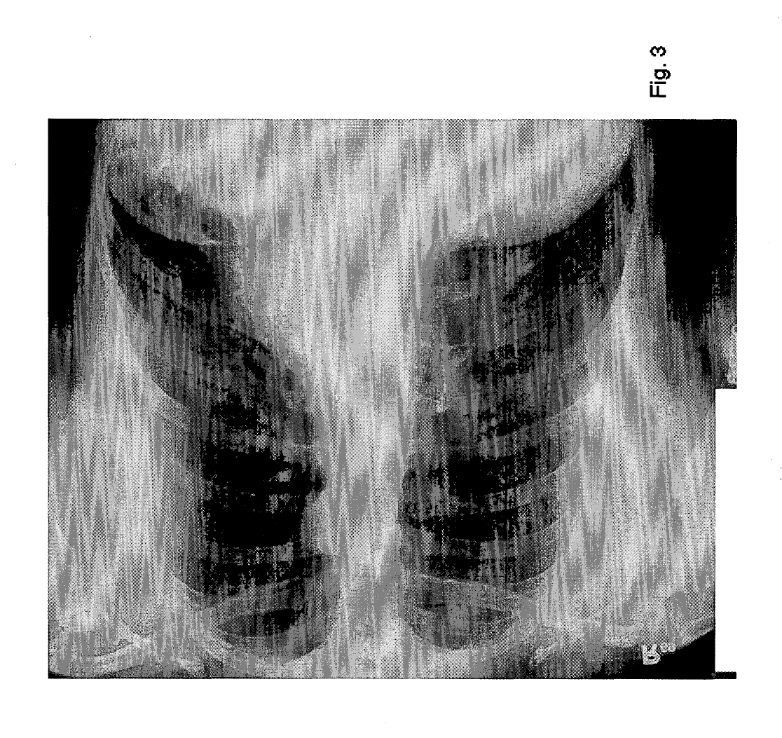

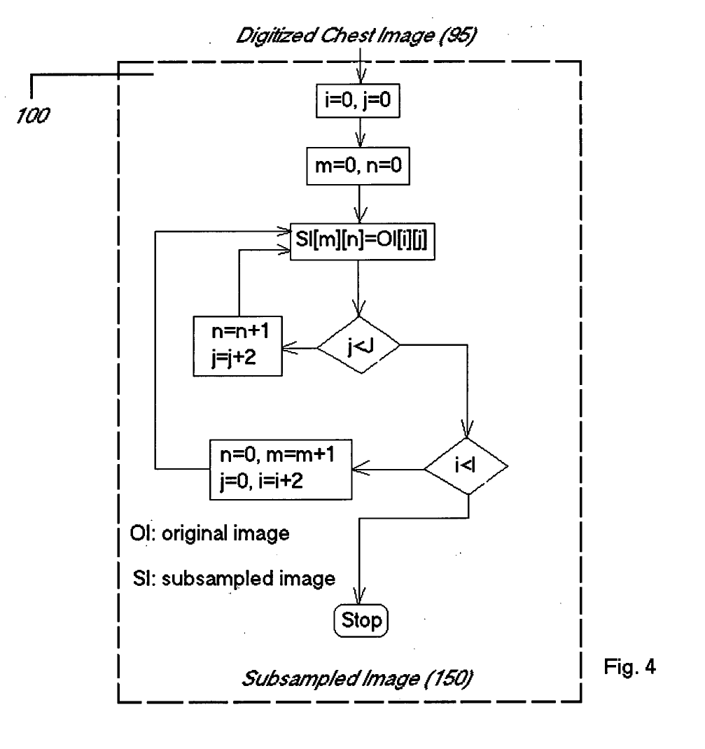

FIG. 1 is a schematic diagram of an embodiment of the invention. First, the digitized image is subsampled using a reduction factor of two to increase the speed of the computational process. This function is included in preprocessing unit 100 of the invention. Thus, an input image (95) of 525×637 will be reduced to an output image (150) of 267×319 after preprocessing. FIG. 2 is a digital chest portrait image of size 525×637. FIG. 3 is a digital chest landscape image of size 525×637. A flow chart of a preferred method for image subsampling is shown in FIG. 4. There, OI (original image) refers to the digital chest image. The I denotes the width of the original image in pixels, and J denotes the height of the original image in pixels.

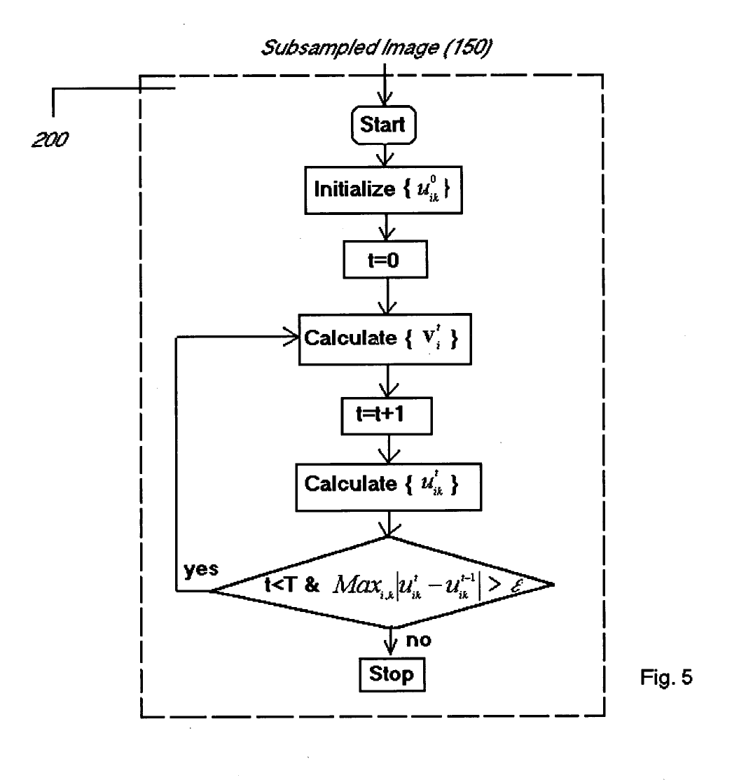

Next is unit 200, the fuzzy clustering unit. According to a preferred embodiment of the invention, in this unit, a Gaussian clustering method (GCM) is employed. Fuzzy clustering is an unsupervised learning technique by which a group of objects is split up into some subgroups based on a measure function. GCM is one of the most commonly used clustering methods. It has a complete Gaussian membership function derived by using a maximum-fuzzy-entropy interpretation. FIG. 5 shows an exemplary flow chart of this method. In FIG. 5,

$${u}_{ik} = exp [ - {‖ {x}_{k} - {v}_{i} ‖}^{2} / 2 {σ}^{2} ] / {\sum }_{j = 1}^{c} exp [ - {‖ {x}_{k} - {v}_{j} ‖}^{2} / 2 {σ}^{2} ] , and {v}_{i} = \\ {\sum }_{k = 1}^{N} {u}_{ik} {x}_{k} / {\sum }_{k = 1}^{N} {u}_{ik} .$$

Here, xₖ represents the k-th input, i.e., k-th pixel, vᵢ represents the center vector of cluster i. uᵢₖ represents membership assignment, that is the degree to which the input k belongs to cluster i. σ is a real constant greater than zero, which represents the “fuzziness” of classification. T represents the maximum number of iterations, ε is a small positive number that determines the termination criterion of the algorithm. N and c represent the number of inputs and number of clusters, respectively. Note that in FIG. 5, the superscripts denote iteration number. After about ten iterations, both of the center vectors and membership function will converge. This method is further described in Li, R. P. and Mukaidono, M., “Gaussian clustering method based on maximum-fuzzy entropy interpretation”, Journal of Fuzzy Sets and Systems, 102 (1999), pp. 253-258, which is incorporated herein by reference. In the present invention, c is 2, which means that the image after clustering is a binary image. Note that a defuzzification process is necessary and is performed by using the following formula:

$${u}_{Ik} = {max}_{i = l}^{i = c} \{ {u}_{ik} ] ∀ k , {u}_{ik} = \{ \begin{matrix}1 & if & i = I \\ 0 & otherwise & \end{matrix}$$

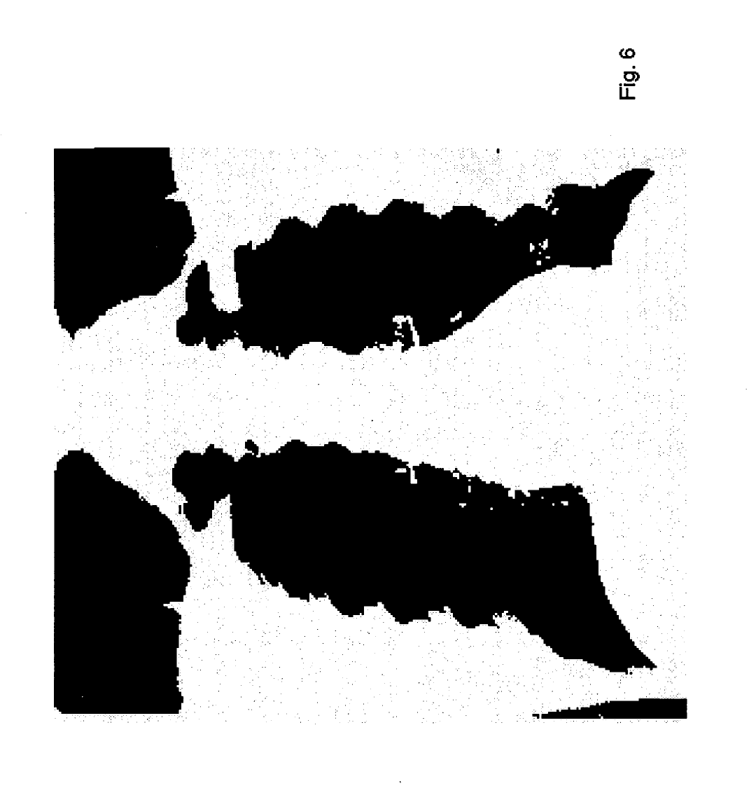

FIG. 6 is the rough image of FIG. 2 obtained through preprocessing unit 100 and fuzzy clustering unit 200. The rough image is a binary image. Pixels in the rough image have two possible gray values, i.e., white or black. Such a binary image roughly presents lung regions (most of the area of black cluster) of the original chest image by contrasting with white cluster area.

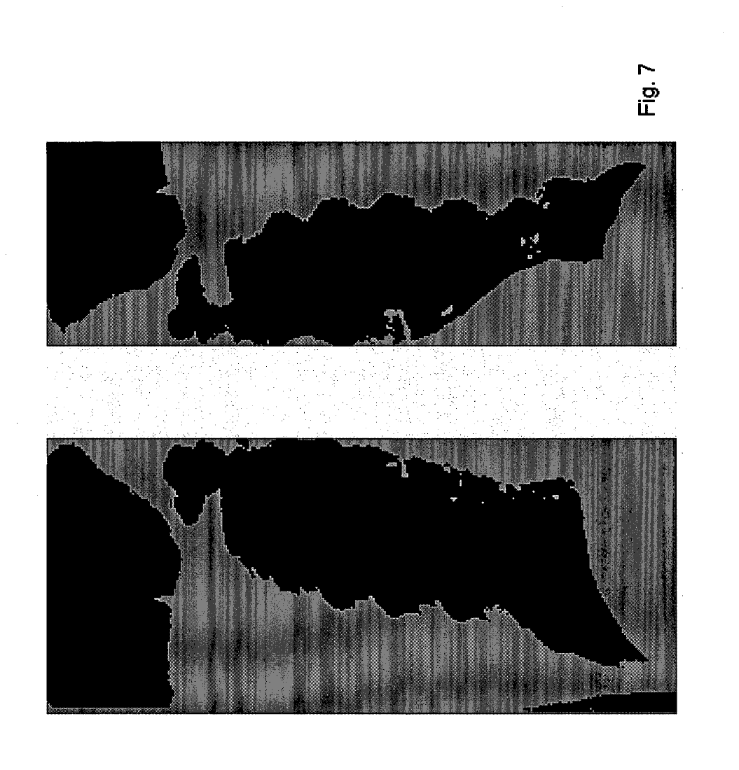

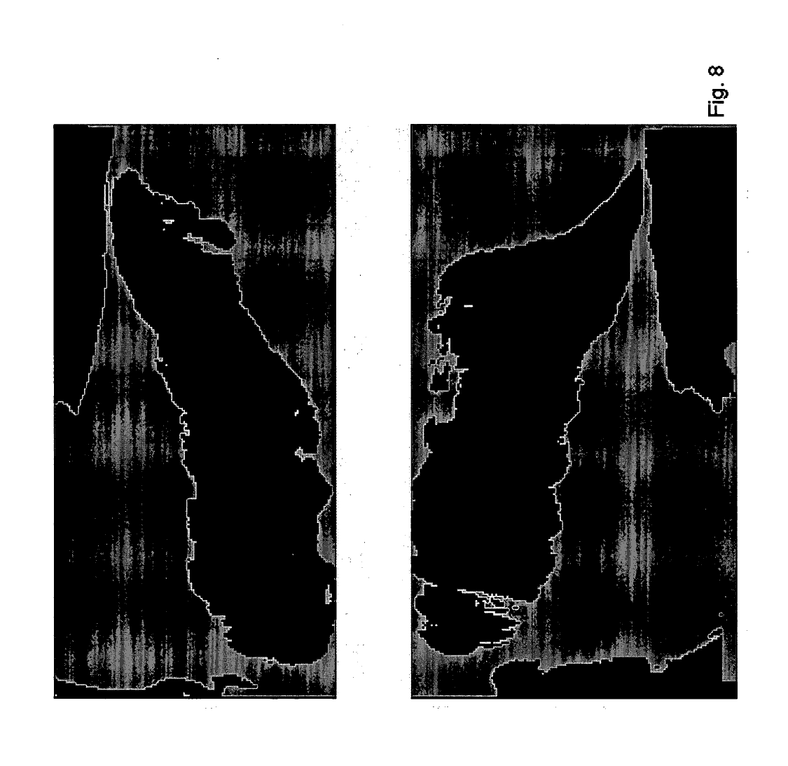

The third unit (300) serves to identify the orientation of a PA chest image. According to the method of the invention, this task is designed to find the orientation of the “spinal” area of a PA chest image. Preferably, the inventive orientation identification method is based on the rough image instead of the original image. Obviously, the difference between portrait and landscape images is that for a portrait image there is a rectangle located in the middle section of the horizontal direction and oriented in the vertical direction, whereas, for a landscape image such a rectangle is located in middle section of the vertical direction and oriented in the horizontal direction. In this rectangle almost all the pixels are of the white gray value. The length of the long side of the rectangle is close to the image's height for the portrait case or close to the image's width for the landscape case. FIG. 7 shows a portrait case, while FIG. 8 shows a landscape case.

The method of identifying orientation of a chest image based on the rough image is simple but effective. The default assumption for the method is that the image is landscape. To judge whether an image is in portrait orientation or not, two conjunctive conditions are used. First, in a portrait image, there is a rectangle as defined above that is located in the middle section of the horizontal direction and oriented in the vertical direction. Further, in a portrait image, gray level value must be black at point (width/4, height/2) and point (3width/4, height/2). Here, “width” represents image width in pixels, and “height” represents image height in pixels. If an image is portrait, it can be passed to post-processing unit (400) directly. Otherwise, a landscape image would be rotated to become a portrait image first, and then passed to the next processing unit. As will be noted below, according to an embodiment of the method of the invention, this rectangle can be used in determining the central zone of a PA chest image.

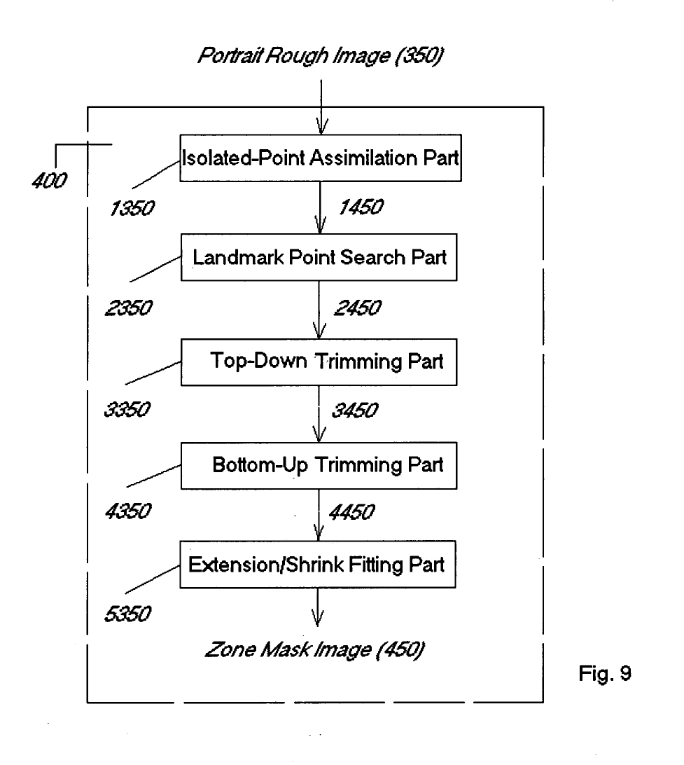

FIG. 9 is a schematic diagram of an embodiment of post-processing unit 400 of FIG. 1. In this unit, there are five (5) functions, as follows: 1) isolated-point assimilation (1350), 2) landmark point search (2350), 3) top-down edge trimming (3350), 4) bottom-up edge trimming (4350), and 5) region extension and/or region shrink (5350).

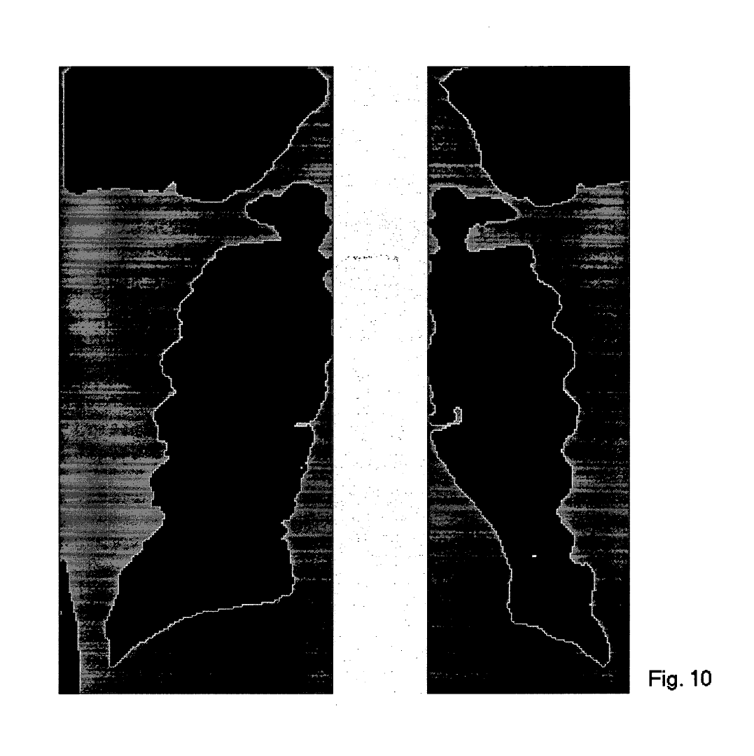

According to an embodiment of the inventive method, the purpose of the isolated-point assimilation part 1350 is to assimilate isolated white points in a black cluster and isolated black points in a white cluster. FIG. 7 is the input (350) of isolated-point assimilation part 1350, and FIG. 10 is the corresponding output (1450) of isolated-point assimilation part 1350. Comparing FIG. 10 to FIG. 7, after this block, isolated points are almost all assimilated.

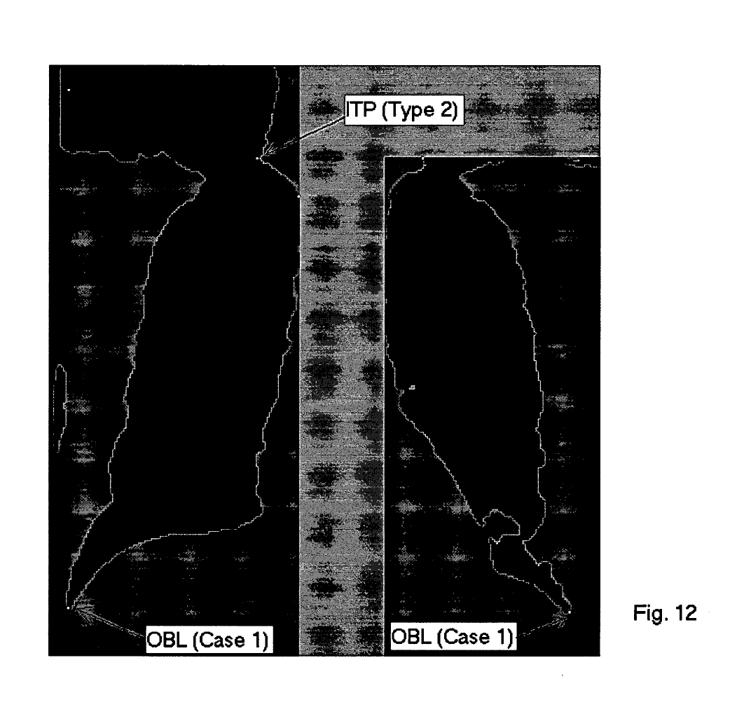

To segment lung regions based on a rough image, the first step is to locate landmark points. Landmark points here include top lung edge points and bottom lung edge points. To determine top lung edge points, rough images are classified into two types. Type 1 images are those in which the boundary of the top lung is clearly separated, as shown in FIG. 7 and FIG. 8. Type 2 images are otherwise rough images, as shown in FIG. 12.

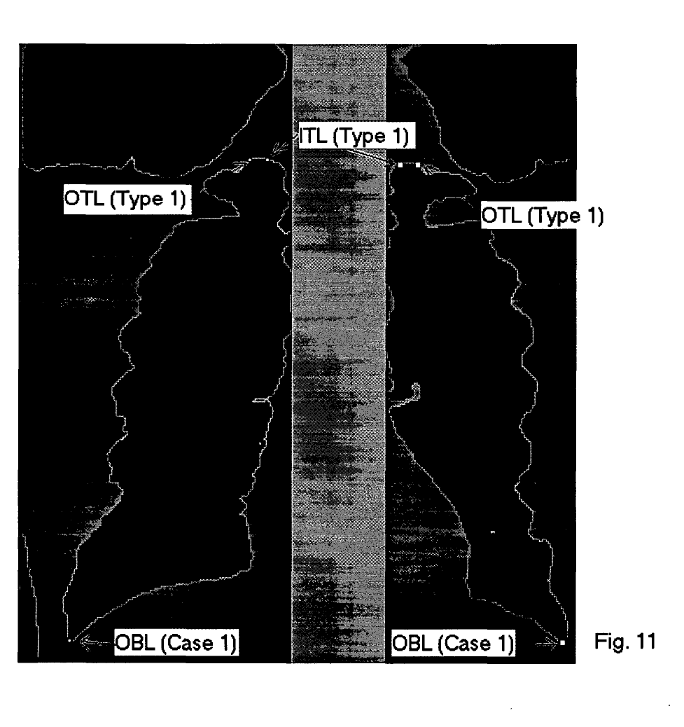

For Type 1, as shown in FIG. 11, the method is straightforward. Considering the right lung, the search region, in the x-direction is from the right side of the rectangular central zone to x=width/4, and in the y direction is from y=15 to y=height/3; note that the point (x,y)=(0,0) is located at the upper left corner of the image. The first pixel encountered that has “black” gray-value is called inner point of top lung (ITL). The final left pixel that has “black” gray-value is called outer point of top lung (OTL). In an exemplary embodiment, the maximum length of top lung edge is set to be 20 pixels. If the top lung edge cannot be found through this process, the rough image is considered to be Type 2. A corresponding process may be carried out for the left lung, as well.

The search process for the top lung edge for Type 2 images is divided into four (4) steps, which will be described in terms of the right lung (i.e., the left side of FIG. 12); corresponding steps may be used to search for the top lung edge of a left lung in a Type 2 image. Step 1 is to find the intermediate y coordinate (y), which is the location of the first pixel whose gray-value is “black” when y decreases to zero from y=height/4 while x=10 (i.e., the value of x is chosen to be close to, but not quite, zero, where zero represents the outer edge of the right lung image). Step 2 is to locate the starting coordinate (x₁, y₁), which must have a gray-value of black and be the nearest such pixel to the left side of the rectangular central zone in the x-direction in the search region y=0 to y′ and x ranging from the left side of the rectangular central zone (i.e., the innermost border of the right lung image) to 0. Step 3 is to locate the ending coordinate (x₂, Y₂), which must have a gray-value of “black” and be the nearest such pixel to the left side of the rectangular central zone in the x-direction in the search region y=y₁ to height/2 and x ranging from the left side of the rectangular central zone to x=width/4. Step 4 is to find the ITL, which must have a gray-value of white and be the furthest such pixel from the left side of the rectangular central zone in the x-direction within the search region y=y₁ to Y₂ and x ranging from the left side of the rectangular central zone to x=width/4. FIG. 12 shows the top lung edge point of a Type 2 image. For this type, the position of OTL is the same as that of ITL.

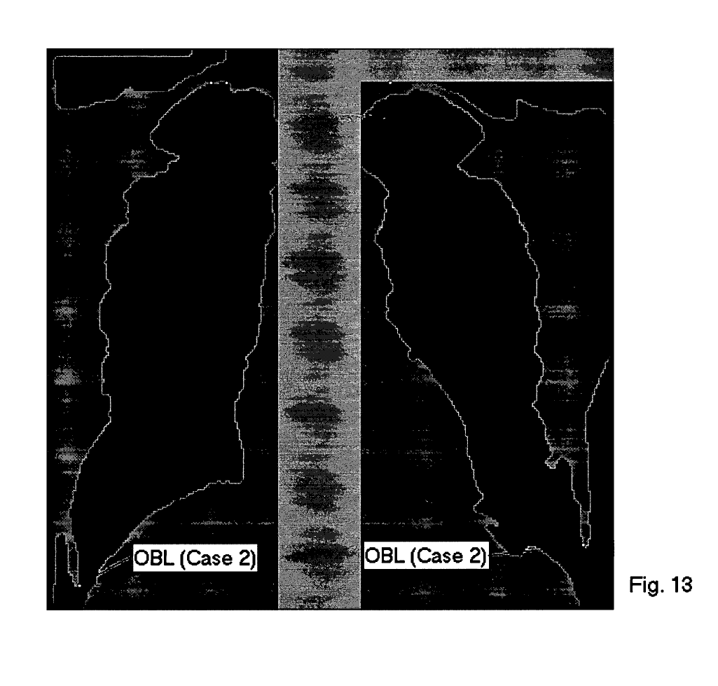

Similarly, for determination of bottom lung edge points, rough images are classified into two (2) cases. Case 1 refers to those in which the boundary of bottom lung is clearly separated as shown in FIG. 7 and FIG. 8. Case 2 are those images that are otherwise rough as shown in FIG. 13. The search region, for the right lung, is from y=height/3 to y=height in the y direction and from x=width/3 to x=0 in the x direction (a corresponding region and process may be applied to the left lung). A common necessary condition of being a bottom lung edge point is that such a point must be an edge point between a “black” region and a “white” region. Let an edge point's coordinates be (x, y). For Case 1, a sufficient condition for being a bottom edge point is: 1) gray-value (gv) of pixel (x-1, y) must be “white”, 2) gv of pixel (x-2, y-1) must be “white”, and 3) gv of pixel (x−1, y+1) must be “white”. In FIG. 11 and FIG. 12, the outer point of bottom lung (OBL) belongs to Case 1. For Case 2, sufficient condition of being bottom edge point is: 1) gray-value (gv) of pixel (x-1, y) must be “white”, 2) gv of pixel (x-1, y-1) must be “white”, and 3) gv of pixel (x−1, y+1) must be “black”. In FIG. 13, OBL belongs to Case 2. Therefore, if the input of landmark point search part (2350) of postprocessing unit (400) in FIG. 9 is an image similar to FIG. 10, then the output will be similar to FIG. 11.

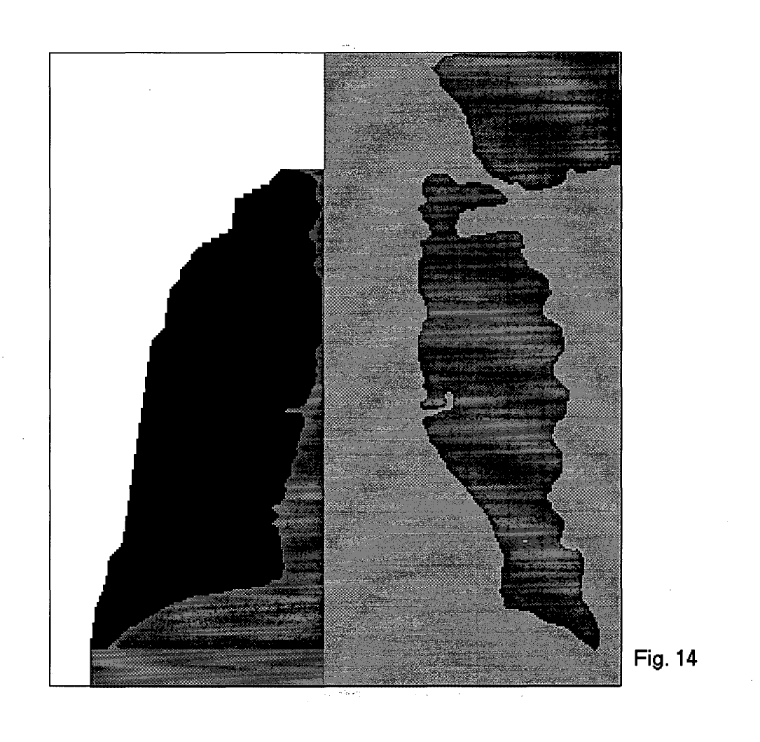

Top-down trimming part (3350) of the post-processing unit (400) in FIG. 9, according to an embodiment of the invention, takes an input image like that shown in FIG. 11, and uses a heuristic rule to trim the boundary of the lung and remove noise. A heuristic rule employed here states that the width of the lung region should continually increase as it moves from top to bottom. Let (xₜ, yₜ) represent the detected outer edge point of the right lung when y=yₜ at evolution time t, and let the successive edge points be (xₜ₊₁, yₜ₊₁), (xₜ₊₂, yₜ₊₂), and so on. According to an embodiment of the invention, if xₜ₊₁>xₜ, then xₜ₊₁ is not changed. Otherwise, xₜ₊₁ reduces 3 pixels every 3 evolution times. The trimming region is from top lung edge point to bottom lung edge point. FIG. 14 shows the result after trimming the right lung shown in FIG. 11. Comparing FIG. 11 with FIG. 14, after top-down trimming, despite the recovery of misclassified bottom lung area and the removal of noise, the boundary of the top lung area is not complete.

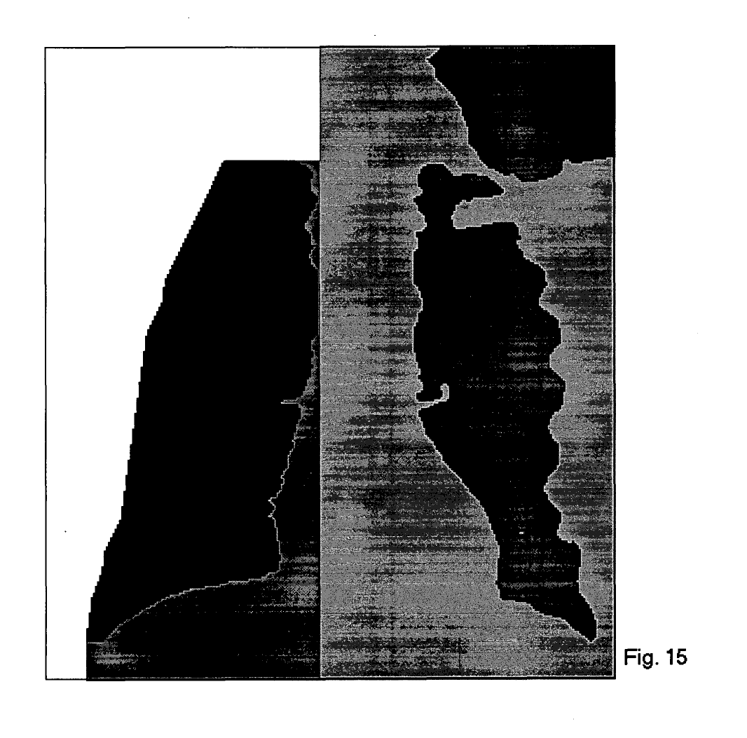

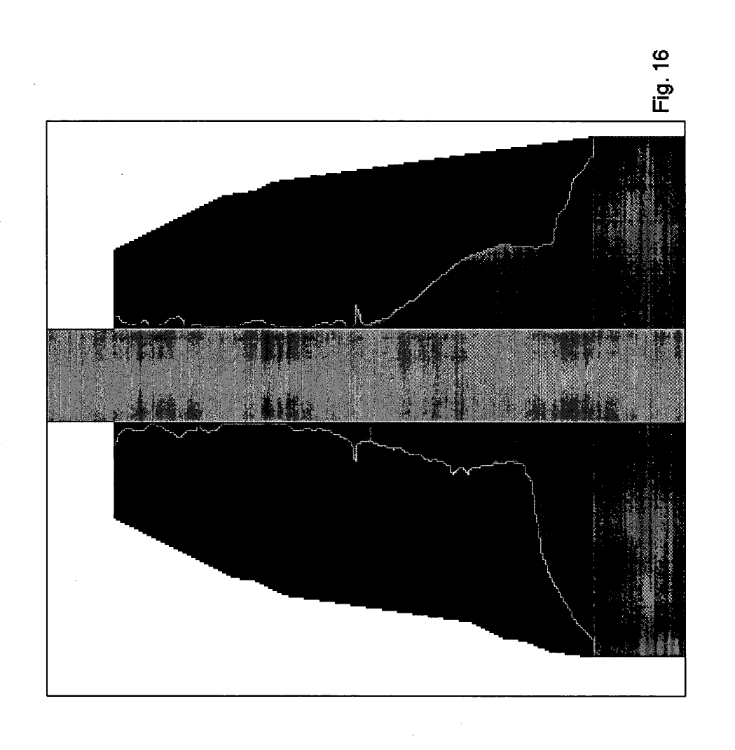

According to an embodiment of the invention, the bottom-up trimming part (4350) of post-processing unit (400) in FIG. 9 is designed to trim the boundary of the top lung area using the following heuristic rule. Like above discussion, let (xₜ, yₜ) represent the detected outer edge point of the right lung when y=yₜ at evolution time t, and let the successive edge points be (xₜ₊₁, yₜ₊₁), (xₜ₊₂, yₜ₊₂), and so on. If xₜ₊₁<xₜ, then xₜ₊₁ is not changed. Otherwise, xₜ₊₁ increases 1 pixel every evolution time. The trimming region is from bottom lung edge point to top lung edge point. FIG. 15 shows the result after bottom-up trimming of the right lung shown in FIG. 14. Similarly, top-down trimming and bottom-up trimming techniques may also be applied to the left lung. Thus, after bottom-up trimming, an initial mask image is obtained as, shown in FIG. 16.

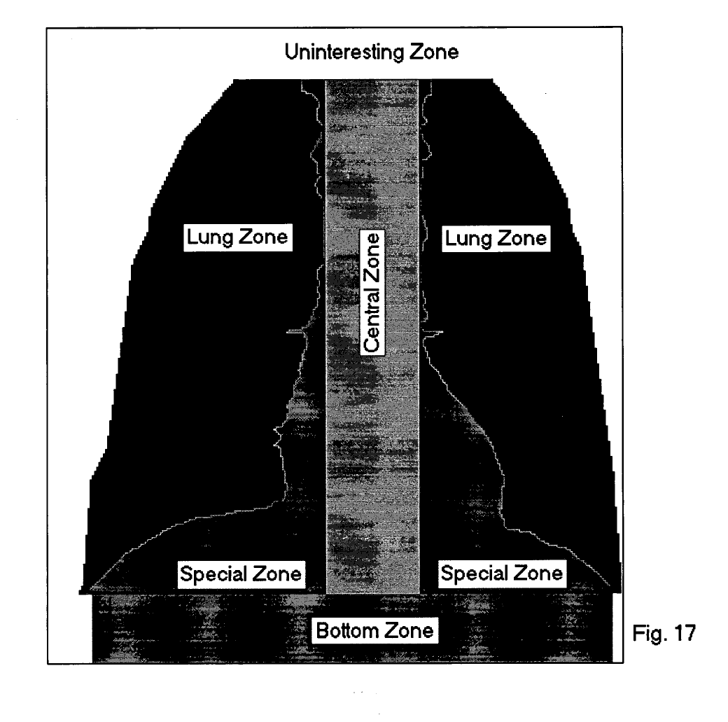

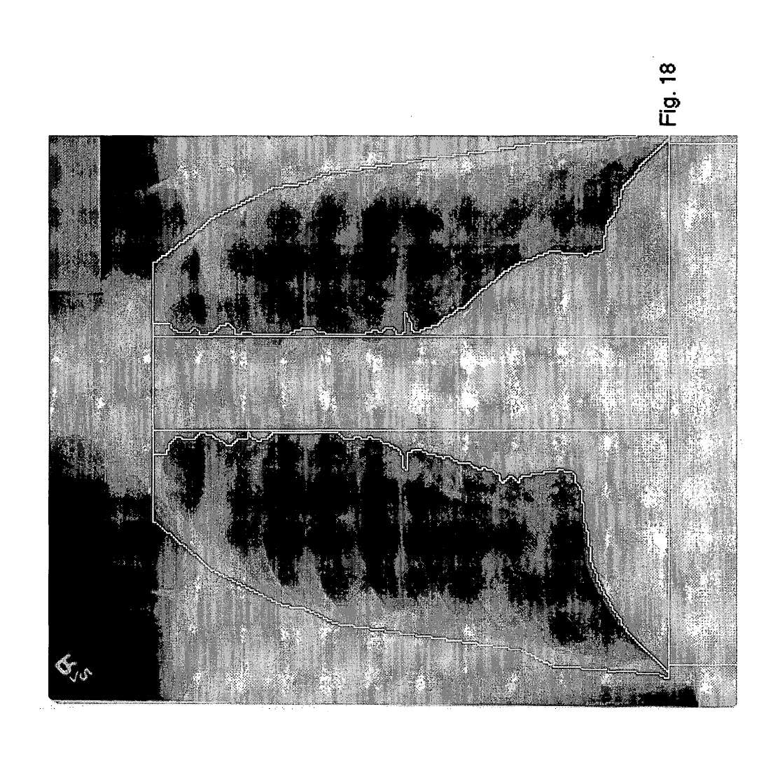

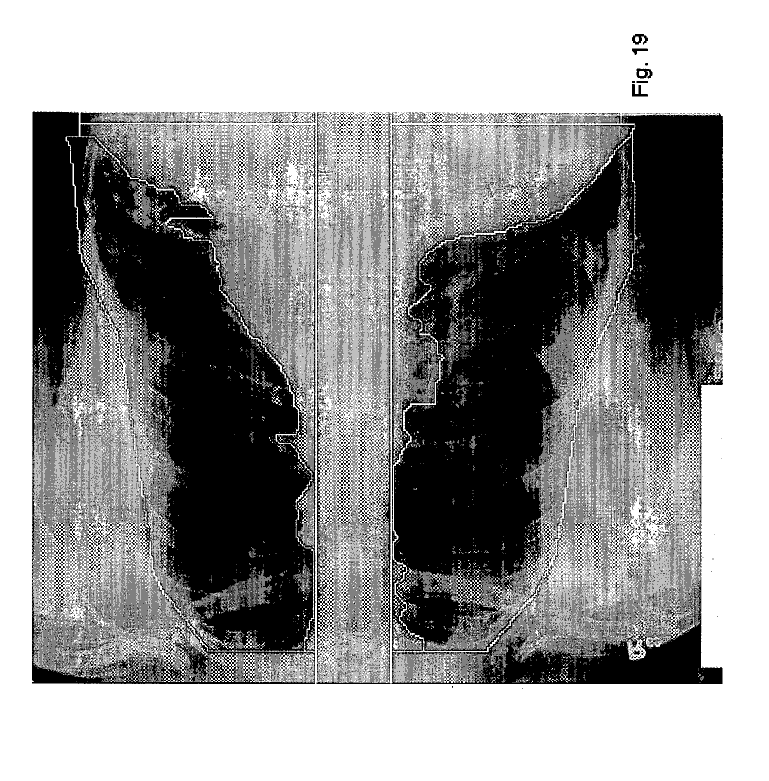

Extension/shrink fitting part (5350) of the post-processing unit (400) in FIG. 9, according to an embodiment of the invention, is designed to adjust the segmented lung region to get the best fit to a real lung. After extension/shrink processing 5350 is completed, a mask that shows five (5) different zones is obtained, as shown in FIG. 17. FIG. 18 shows the chest image (portrait image) of FIG. 2 overlaying boundaries of the zone mask image of FIG. 17. FIG. 19 shows a chest image (landscape image) of FIG. 3 overlaying boundaries of a corresponding zone mask image. The five zones cover the following anatomic regions:
Lung Zone: left lung and right lung;
Central Zone: superior mediastinum, heart, and part of subdiaphragm;
Special Zone: part of lung, part of heart, and part of subdiaphragm;
Bottom Zone:. most of subdiaphragm;
Uninteresting Zone: background, base of neck, and axilla.

Table 1 illustrates the chest image orientation identification performance of the method for 3459 images. Of them, 519 images were landscape. images, and the rest were portrait images.

TABLE 1

| | Images | Images | |
|---|---|---|---|
| Number of Images | Recognized | Missed | Identification Rate |
| 519 (landscape) | 512 | 7 | 98.6% |
| 2940 (portrait) | 2940 | 0 | 100% |

Table 2 illustrates the rib-cage detection performance of the method for 3459 chest images.

TABLE 2

| Category | Number of Images | Percentage |
|---|---|---|
| good | 3215 | 92.9% |
| fair | 149 | 4.3% |
| bad | 50 | 1.4% |
| quit | 45 | 1.3% |

It should be noted that, as in any ill-defined problem, the evaluation criterion used here is very subjective. The “quit” case indicates that the method as embodied for these trials was unable to deal with a given image.

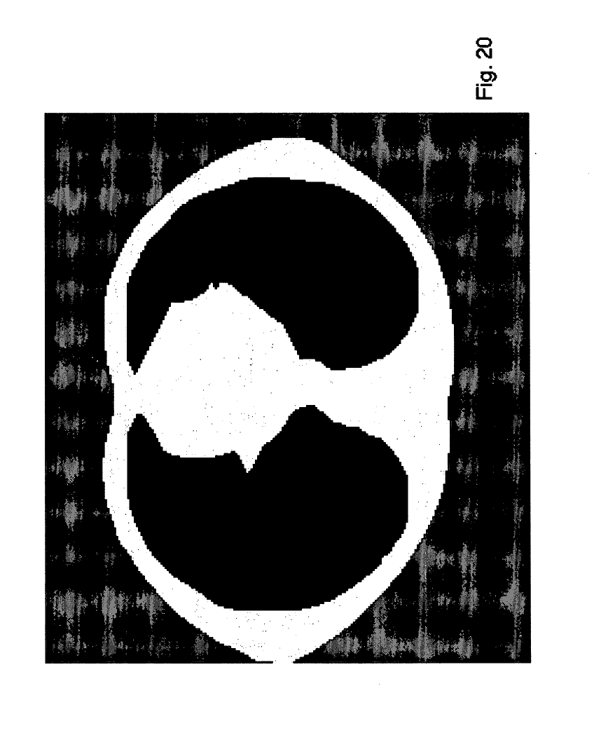

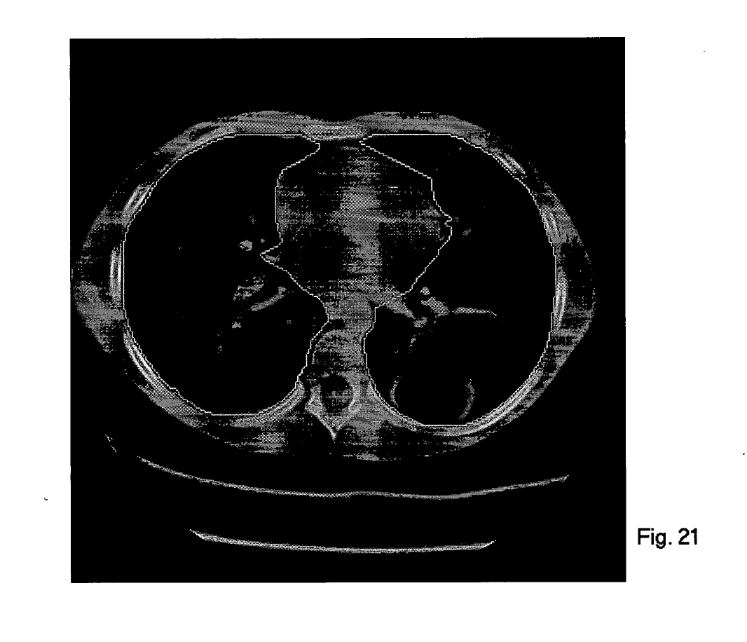

The same concept has been expanded to lung segmentation in a CT image. FIGS. 20-21 demonstrate the performance of applying the invention to a CT image.

Obviously, numerous modifications to and variations of the present invention are possible in light of the above technique. It is, therefore, to be understood that within the scope of the appended claims, the invention may be implemented in situations other than as specifically described herein. Although the present application is focused on chest image and CT image, the concept can be expanded to other medical images and other object segmentation problems, such as MRI, brain and vessel segmentation, and the like. The invention is thus of broad application and not limited to the specifically disclosed embodiment.

1. A method for identifying the orientation of an interesting object (10) in a digital medical image, the method comprising the steps of:
a) creating a rectangular interesting image mask that covers said interesting object from original digital medical image;
b) generating a rough image based on said interesting image mask, the rough image coarsely describing the interesting object; and
c) identifying the orientation of said interesting object based on the rough image.

2. The method of claim 1, wherein said interesting object is an anatomical region.

3. The method of claim 1, wherein said interesting image mask is one of:
manually selected by a user; automatically selected by a program; and
generated by another system.

4. The method of claim 1, wherein the size of the interesting image mask is the same as that of the digital medical image.

5. The method of claim 1, wherein said rough image is a binary image, and wherein said step of generating a rough image comprises the step of using unsupervised learning techniques to segment said interesting object.

6. The method of claim 5, wherein said step of using unsupervised learning techniques further includes the steps of:
using a clustering technique;
using a thresholding technique; and
using a self-organizing technique.

7. The method of claim 1, further including the use of one or more heuristic rules.

8. The method of claim 7, wherein the one or more heuristic rules are used in the step of identifying the orientation of the interesting object, and wherein the one or more heuristic rules compare features extracted from said rough image.

9. A system that performs identification of the orientation of an interesting object in digital medical image, the system comprising:
a digitizer system;
a computer system; and
a computer-readable medium containing software implementing the method of claim 1.

10. A method for segmenting interesting objects (10) in digital medical images, the method comprising the steps of:
a) creating a rectangular interesting image mask that covers said interesting object from an original digital medical image;
b) generating a rough image based on said interesting image mask, the rough image coarsely describing said interesting object; and
c) performing a post-process on said rough image.

11. The method of claim 10, wherein said interesting object is an anatomical region.

12. The method of claim 10, wherein said interesting image mask is one of:
manually selected by a user; automatically selected by a program; and
generated by another system.

13. The method of claim 10, wherein the size of the interesting image mask is the same as that of original medical image.

14. The method of claim 10, wherein said rough image is a binary image, and wherein said step of generating a rough image comprises using unsupervised learning techniques to segment said interesting object.

15. The method of claim 14, wherein said step of using unsupervised learning techniques further includes the steps of:
using a clustering technique;
using a thresholding technique; and
using a self-organizing technique.

15. The method of claim 10, wherein said step of performing a post-process comprises the steps of:
a) searching landmark points; and
b) trimming a boundary and removing noise.

16. The method of claim 10, wherein said post-process is based upon the rough image.

17. The method of claim 16, wherein said step of searching landmark points includes at least one of the steps of:
searching top edge points and bottom edge points of the interesting object; and
searching left edge points and right edge points of the interesting object.

18. The method of claim 16, wherein said step of trimming a boundary and removing noise further includes:
(a) searching edge points of the interesting object; and
(b) using one or more heuristic rules.

19. The method of claim 18, wherein a region of searching edge points used in said step of searching edge points is from top edge point to bottom edge point in the vertical direction, and from left edge point to right edge point in the horizontal direction.

20. The method of claim 18, wherein the one or more heuristic rules used in the step of trimming boundary and removing noise include the steps of:
using common logic inference; and
comparing the interesting object in the rough image with a real object.

21. A system for segmenting interesting objects (10) in digital medical images, the system comprising:
a digitizer system;
a computer system; and
a computer-readable medium containing software implementing the method of claim 10.
